(19) United States
(12) Patent Application Publication
(10) Pub. No.: US 2023/0335225 A1
(43) Pub. Date: Oct. 19, 2023

(54) METHOD AND SYSTEM FOR VIRTUAL INSPECTION AND SIMULATION OF RARE EARTH PRODUCTION PROCESS

(71) Applicant: EAST CHINA JIAOTONG UNIVERSITY, NANCHANG CITY (CN)

(72) Inventors: HUI YANG, NANCHANG CITY (CN); ZHIQIN KUANG, NANCHANG CITY (CN); JIANYONG ZHU, NANCHANG CITY (CN); RONGXIU LU, NANCHANG CITY (CN); FANGPING XU, NANCHANG CITY (CN)

(21) Appl. No.: 17/870,685

(22) Filed: Jul. 21, 2022

(30) Foreign Application Priority Data
Apr. 14, 2022 (CN) 202210390469.5

(51) Int. Cl. G16C 20/10

(52) U.S. Cl. CPC G16C 20/10

(57) ABSTRACT

The present disclosure relates to a method and system for virtual inspection and simulation of a rare earth production process, and belongs to the field of digital twin technologies. According to the method, a virtual workshop is built based on a geometric model and a control script of a production site as well as real-time data of the site; and simulation of an entire process is completed. Visualized demonstration of data in each production process is realized by building a virtual rare earth workshop and establishing a data connection between an actual workshop and the virtual rare earth workshop. In addition, fast inspection of a production device is realized. In addition, contents of components are forecast based on historical data according to an extraction mechanism; and whether to give a warning is determined automatically based on the contents. Therefore, real-time and precise forecasting of a process index is realized.

### CROSS REFERENCE TO RELATED APPLICATION

This patent application claims the benefit and priority of Chinese Patent Application No. 202210390469.5, filed on Apr. 14, 2022, the disclosure of which is incorporated by reference herein in its entirety as part of the present application.

### TECHNICAL FIELD

The present disclosure belongs to the field of digital twin technologies, and in particular, to a digital twin-based method and system for virtual inspection and simulation of a rare earth production process.

### BACKGROUND ART

Because the enrichment degree of rare earth in a raw material is low, and an extraction process having dozens or hundreds of stages needs to be performed to obtain a target product with high purity, the entire extraction process has a large delay, is complex to control, and wastes time and labor in working condition detection. In addition, centralized optimization and inspection are hard to implement because data of each process is independent. Because the extraction process has a large delay, it is critical to perform real-time working condition forecasting and timely optimal control.

However, an existing rare earth process relies heavily on manual control and inspection, which makes process inspection difficult, and causes a slow response to an abnormal working condition. There are also the following problems: Process optimization relies heavily on manual work; and it is difficult to utilize on-site data.

Therefore, there is an urgent need in the art for a technical solution capable of implementing centralized monitoring and controlling of process indexes and simulating and forecasting a process index based on on-site data.

### SUMMARY

An objective of the present disclosure is to provide a digital twin-based method and system for virtual inspection and simulation of a rare earth production process. According to the method, a virtual workshop is built first; a one-to-one correspondence is established between the virtual workshop and real-time data; and simulation of an entire process is completed. Therefore, automatically inspection is implemented. In addition, contents of components are forecast based on historical data according to an extraction mechanism; and whether to give a warning is determined automatically based on the contents. Therefore, the following problems are effectively resolved: An existing technology relies heavily on manual control and inspection, which makes process inspection difficult, and causes a slow response to an abnormal working condition. Process optimization relies heavily on manual work. It is difficult to utilize on-site data.

To achieve the above objective, the present disclosure provides the following technical solutions:

The present disclosure provides a method for virtual inspection and simulation of a rare earth production process. The method includes the following steps:

obtaining real-time data of a production site, where the real-time data includes data of a process production index;

building a virtual rare earth workshop based on a geometric model and a control script of the production site as well as the real-time data, for a user to perform inspection;

obtaining historical data of a process production index;

optimizing an extraction mechanism model based on the historical data of the process production index according to a parameter optimization algorithm to obtain a data-driven model, where the extraction mechanism model is a mathematical model representing an extraction mechanism of rare earth;

obtaining process information input by the user;

forecasting a content of each element in a finished product of a process based on the process information by using the data-driven model; and

determining, based on the content of each element, whether to give a warning.

In some examples, the optimizing an extraction mechanism model based on the historical data of the process production index according to a parameter optimization algorithm to obtain a data-driven model specifically includes:

optimizing a separation coefficient in the extraction mechanism model based on the historical data of the process production index according to a particle swarm optimization algorithm, to obtain the data-driven model.

The present disclosure further discloses a system for virtual inspection and simulation of a rare earth production process. The system includes: a control information system for rare earth production, a virtual workshop for rare earth production, and a digital twin service system, where

the control information system for rare earth production is configured to obtain real-time data of a production site, and control a production process;

the virtual workshop for rare earth production is configured to build a virtual rare earth workshop based on a geometric model and a control script of the production site as well as the real-time data, for a user to perform inspection;

the digital twin service system is configured to:

obtain historical data of a process production index;

optimize an extraction mechanism model based on the historical data of the process production index according to a parameter optimization algorithm, to obtain a data-driven model, where the extraction mechanism model is a mathematical model representing an extraction mechanism of rare earth;

obtain process information input by the user;

forecast a content of each element in a finished product of a process based on the process information by using the data-driven model; and

determine, based on the content of each element, whether to give a warning.

In some examples, the control information system for rare earth production includes a basic control module and process detection modules, where

the basic control module is configured to execute a control instruction;

the basic control module includes a motor converter, a variable-flow pump, a metering pump, a solenoid valve, and a PLC, where the motor converter is configured to adjust a rotational speed of an agitator; the variable-flow pump and the metering pump are configured to control quantitative feeding in an extraction process; the solenoid valve is configured to control feeding and discharging of a feed solution; and the PLC is configured to obtain a control instruction, and transmit the control instruction to the motor converter, the variable-flow pump, the metering pump, and the solenoid valve;

the process detection module is configured to obtain real-time data; and

the process detection module includes a flowmeter, a level gauge, a thermometer, a pH meter, and component content detection devices, where the flowmeter is configured to monitor and control a feeding flow rate and a discharging flow rate of the feed solution in an extraction process; the level gauge is configured to monitor and detect levels of liquid in an extraction tank and a storage tank; the thermometer and the pH meter are configured to prepare a scrubbing solution and an extraction solution that meet requirements for temperature and potential of hydrogen; and the component content detection devices are disposed for each detection level of a rare earth extraction process.

In some examples, the system further includes:

a twin database configured to store historical data, real-time data, and warning information.

In some examples, the control information system for rare earth production further includes a data transmission module configured to exchange data between the twin database and the control information system for rare earth production.

In some examples, the virtual workshop for rare earth production includes: a data exchanging module, a geometric model base, a user interaction module, and a scene changing module, where

the data exchanging module is configured to regularly query related data in the twin database according to process-data correspondence;

the geometric model base is configured to build a virtual rare earth workshop, and visualize the virtual rare earth workshop, where the virtual rare earth workshop is built by using modeling software;

the user interaction module is configured to control a scene viewing angle when the user performs inspection, and a demo animation; and

the scene changing module is configured to change a virtual rare earth workshop when the user performs inspection.

In some examples, the digital twin service system further includes a process optimization module configured to:

obtain an optimization strategy by using an optimal control algorithm based on an objective set by the user, where the optimal control algorithm is used for optimal control over a flow rate of a reagent in extraction based on static setting and dynamic compensation.

In some examples, the controlling a production process specifically includes:

controlling the production process according to the optimization strategy.

In some examples, the digital twin service system further includes a model updating module configured to:

calculate an error between the forecast content and actual data of a process index, to obtain a content error; and

adjust the data-driven model based on the content error according to the parameter optimization algorithm.

According to the specific examples provided by the present disclosure, the present disclosure discloses the following technical effects:

According to the method, a virtual workshop is built based on a geometric model and a control script of a production site as well as real-time data of the site; and simulation of an entire process is completed. Visualized demonstration of data in each production process is realized by building a virtual rare earth workshop and establishing a data connection between an actual workshop and the virtual rare earth workshop. In addition, fast inspection of a production device is realized. In addition, contents of components are forecast based on historical data according to an extraction mechanism; and whether to give a warning is determined automatically based on the contents. Therefore, real-time and precise forecasting of a process index is realized. In addition, a response is given fast to a change of a production condition, and warning and optimizing data is provided for the system, such that a burden of an operator is reduced, control over a production process is optimized, production and management efficiency is improved, and process data of the site is utilized effectively.

### BRIEF DESCRIPTION OF THE DRAWINGS

To describe the technical solutions in the examples of the present disclosure or in the prior art more clearly, the following briefly introduces the accompanying drawings required for describing the examples. Apparently, the accompanying drawings in the following description show merely some examples of the present disclosure, and a person of ordinary skill in the art may still derive other drawings from these accompanying drawings without creative efforts.

FIG. 1 is a flowchart of a method for virtual inspection and simulation of a rare earth production process according to Example 1 of the present disclosure;

FIG. 2 is a block diagram of a system for virtual inspection and simulation of a rare earth production process according to Example 2 of the present disclosure;

FIG. 3 is a block diagram of a digital twin-based system for virtual inspection and simulation of a rare earth production process according to Example 4 of the present disclosure;

FIG. 4 is a schematic flowchart of a logic of a control information system for rare earth production according to Example 4 of the present disclosure;

FIG. 5 is a schematic flowchart of a logic of a twin database according to Example 4 of the present disclosure;

FIG. 6 is a schematic flowchart of a logic of a digital twin service system according to Example 4 of the present disclosure; and

FIG. 7 is a schematic flowchart of a logic of a process index forecasting model according to Example 4 of the present disclosure.

### DETAILED DESCRIPTION OF THE EMBODIMENTS

The technical solutions of the examples of the present disclosure are clearly and completely described below with reference to the accompanying drawings. Apparently, the described examples are merely a part rather than all of the examples of the present disclosure. All other examples obtained by those of ordinary skill in the art based on the examples of the present disclosure without creative efforts shall fall within the protection scope of the present disclosure.

An existing rare earth process relies heavily on manual control and inspection. As a result, there is an urgent need for a system for virtual inspection and simulation of a rare earth production process. The following uses a multi-component cascade extraction-based separation process as an example. In an existing rare earth extraction process, a process index, namely, a content of each element, is obtained through an off-line test for a detection-level solution, such that an abnormal working condition is prevented, and a specified value of a controller is further adjusted based on the process index to optimize a production process. An existing rare earth production process has the following problems: Process inspection is difficult; a response to an abnormal working condition is slow; process optimization relies heavily on manual work; and it is difficult to utilize on-site data. To resolve the above problems, the present disclosure designs an overall framework that monitors, controls, and processes on-site data in a centralized manner, stores and exchanges data in a database, performs simulation, state monitoring and controlling, and free inspection in a virtual scene, and implements integration of all modules and a human-computer interaction function in a twin service system for rare earth. Digital twin may be considered as an integration of a model and an algorithm, is capable of implementing functions of data visualization, forecasting, and process optimization, and is mainly used for reducing a burden of an operator, and improving production efficiency.

An objective of the present disclosure is to provide a digital twin-based method and system for virtual inspection and simulation of a rare earth production process. According to the method, a virtual workshop is built first; a one-to-one correspondence is established between the virtual workshop and real-time data; and simulation of an entire process is completed. Therefore, automatically inspection is implemented. In addition, contents of components are forecast based on historical data according to an extraction mechanism; and whether to give a warning is determined automatically based on the contents. Therefore, the following problems are effectively resolved: An existing technology relies heavily on manual control and inspection, which makes process inspection difficult, and causes a slow response to an abnormal working condition. Process optimization relies heavily on manual work. It is difficult to utilize on-site data.

To make the above objectives, features, and advantages of the present disclosure clearer and more comprehensible, the present disclosure will be further described in detail below with reference to the accompanying drawings and specific examples.

### EXAMPLE 1

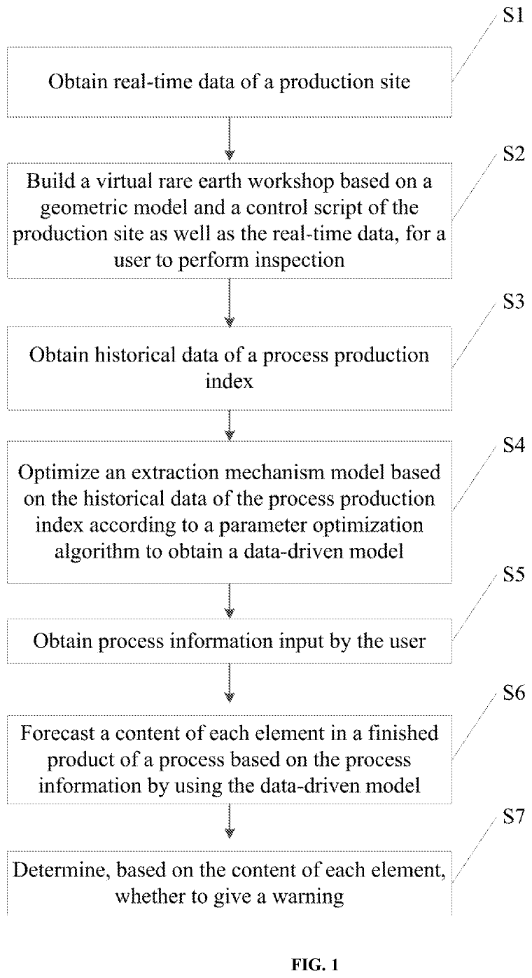

As shown in FIG. 1, this example provides a method for virtual inspection and simulation of a rare earth production process. The method includes the following steps.

S1: Obtain real-time data of a production site, where the real-time data includes data of a process production index.

The data of the process production index includes a feeding flow rate and a discharging flow rate of a feed solution in an extraction process, levels of liquid in an extraction tank and a storage tank, temperatures and pH values of a scrubbing solution and an extraction solution as well as a content of each component of a substance at each detection level.

S2: Build a virtual rare earth workshop based on a geometric model and a control script of the production site as well as the real-time data, for a user to perform inspection.

First, a virtual workshop is built based on the geometric model and the control script of the production site; and then, the above real-time data is configured to the virtual workshop piece by piece, such that the user can perform free inspection. The virtual workshop may be built based on an actual production device by using modeling software such as 3DMAX and MAYA.

S3: Obtain historical data of a process production index.

The historical data of the process production index may be manually input, or may be obtained after the above real-time data is stored.

S4: Optimize an extraction mechanism model based on the historical data of the process production index according to a parameter optimization algorithm to obtain a data-driven model.

The extraction mechanism model is a mathematical model representing an extraction mechanism of rare earth, and includes models of separation coefficients between extraction stages (for example, formulas (1)-(3)), formulas for conservation of materials and the like (for example, formula (4)-(7)), and models of parameters for compensating a separation coefficient (for example, formula (8)-(9)).

$${β}_{( i + 1 ) / i} = \frac{{Y}_{i + 1} \times {X}_{i}}{{Y}_{i} \times {X}_{i + 1}} \qquad ( 1 )$$

$${β}_{1 / i} = \frac{{Y}_{i} \times {X}_{1}}{{Y}_{1} \times {X}_{i}} \qquad ( 2 )$$

$${β}_{i / N} = \frac{{Y}_{N} \times {X}_{i}}{{Y}_{i} \times {X}_{N}} \qquad \text{(3)}$$

$${f}_{B}^{′} \times {X}_{[ 1 , i ]} = W \times {X}_{[ k + 1 , i ]} - \overline{S} \times {Y}_{[ k , i ]} , i = 1 , 2 , … , N ; k = 1 , 2 , … n \qquad ( 4 )$$

$${X}_{[ k + 1 , i ]} = ( \overline{S} \times {Y}_{[ k , i ]} + {f}_{B}^{′} \times {X}_{[ 1 , i ]} ) / W \qquad ( 5 )$$

$${\overline{f}}_{A}^{′} \times {Y}_{[ n + m , i ]} = ( \overline{S} + 1 ) \times {Y}_{[ k + 1 , i ]} - W \times {X}_{[ k , i ]} , \qquad ( 6 )$$

$$i = 1 , 2 , … , N ; k = 1 , 2 , … , m$$

$${Y}_{[ k + 1 , i ]} = ( W \times {X}_{[ k , i ]} + {\overline{f}}_{A}^{′} \times {Y}_{[ n + m , i ]} ) / ( \overline{S} + 1 ) \qquad ( 7 )$$

$${Y}_{i} = ( {X}_{i} \times {\prod }_{i = 1}^{N} K . * {β}_{1 / i} ) / {\sum }_{i = 1}^{N} ( {X}_{i} \times {\prod }_{i = 1}^{N} K . * {β}_{1 / i} ) \qquad \text{(8)}$$

$${X}_{i} = ( {Y}_{i} \times {\prod }_{i = 1}^{N} K . * {β}_{i / N} ) / {\sum }_{i = 1}^{N} ( {Y}_{i} \times {\prod }_{i = 1}^{N} K . * {β}_{i / N} ) \qquad ( 9 )$$

Formula (1) denotes a relationship between an organic component content and a separation coefficient for two adjacent elements. Formula (2) denotes a separation coefficient of an i^{th} element relative to a first element. Formula (3) denotes a separation coefficient of the i^{th} element relative to a last element. Formulas (4 and 5) denote relationships between aqueous components of two adjacent levels in an extraction stage. Formulas (6 and 7) denote relationships between organic components of two adjacent levels in a scrubbing stage. Formulas (8 and 9) denote relationships between an aqueous component and an organic component that are of a same level and have been compensated based on a separation coefficient.

X denotes a content of an aqueous component. Y denotes a content of an organic component. Conservation of materials between extraction stages is used to describe conservation of contents of extraction elements in two adjacent extraction stages. f_{F} in formula (4) of a conservation of materials model denotes a content of the feed solution. f′_{B} denotes an output mole fraction of a hardly extractable component. f̅′_{A} denotes an output mole fraction of an easily extractable component. An organic composition of output component A and an aqueous composition of output component B are obtained by dividing f̅′_{A} and f̅′_{B} into a content of output component A and a content of output component B, respectively. S̅ denotes an extraction capacity. W denotes a scrubbing capacity. i denotes the number of an extracted element. i ranges from 1 to N. N denotes the number of an element that is extracted last. Yi denotes a content of an organic component of the i^{th} element. Xi denotes a content of an aqueous component of the i^{th} element. β_{(1+i)/i} denotes a separation coefficient of an element relative to a next element. β_{1/i} denotes a separation coefficient of the i^{th} element relative to the first element. β_{i/N⋅} denotes a separation coefficient of the last element relative to the i^{th} element. X_{[k+1,i]} denotes a content of an aqueous component that is of a (k+1)^{th} level and of the i^{th} element. Y_{[k,i]} denotes a content of an aqueous component that is of a k^{th} level and of the i^{th} element. A conservation of materials relationship of the hardly extractable component may be established among a product of the output mole fraction of the hardly extractable component f′_{B} and a content of an aqueous component, contents of aqueous and organic components of a feeding level, the extraction capacity, and the scrubbing capacity. Similarly, a conservation of materials relationship of the easily extractable component may be established among a product of the output mole fraction of the easily extractable component f̅′_{A} and a content of an organic component, contents of aqueous and organic components of a feeding level, the extraction capacity, and the scrubbing capacity. Contents of components of all levels can be deduced by level according to these conservation of materials relationships. A case in which on-site extraction is insufficient can be simulated by adding a compensation coefficient K to a separation coefficient. Then, a relationship between an aqueous component and an organic component can be obtained by performing deformation. A content of one of the two components can be obtained based on a content of the other component.

Second, a separation coefficient in the extraction mechanism model is optimized based on the historical data of the process production index according to a particle swarm optimization algorithm, to obtain the data-driven model. An optimization manner is optimizing the compensation parameter K of the separation coefficient according to an improved particle swarm optimization algorithm, to obtain an optimal compensation parameter.

S5: Obtain process information input by the user.

S6: Forecast a content of each element in a finished product of a process based on the process information by using the data-driven model. In this example, contents of three elements Ce, Pr, and Nd are mainly forecast based on the process information input by the user.

S7: Determine, based on the content of each element, whether to give a warning.

According to the present disclosure, based on a real-time process index forecasting model built in a digital twin system, real-time and precise forecasting of a process index can be implemented by using a real-time process index forecasting module. In addition, a response is given fast to a change of a production condition, and warning and optimizing data is provided for the system, such that a burden of an operator is reduced, control over a production process is optimized, production and management efficiency is improved, and process data of the site is utilized effectively.

According to the present disclosure, visualized demonstration of data in each production process is realized by building a virtual rare earth workshop and establishing a data connection between an actual workshop and the virtual rare earth workshop. In addition, fast inspection of a production device is realized.

A control strategy is optimized based on an optimization objective specified by the user and actual process index data by using a process optimization module in a digital twin service system, thereby optimizing the production process.

### EXAMPLE 2

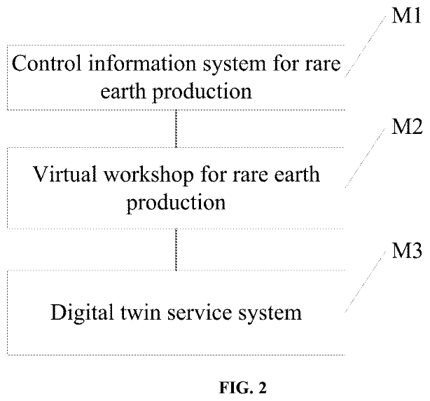

As shown in FIG. 2, this example provides a system for virtual inspection and simulation of a rare earth production process. The system includes: a control information system M1 for rare earth production, a virtual workshop M2 for rare earth production, and a digital twin service system M3.

The control information system M1 for rare earth production is configured to obtain real-time data of a production site, and control a production process.

The control information system M1 for rare earth production includes a basic control module and a process detection module.

The basic control module is configured to execute a control instruction.

The basic control module includes a motor converter, a variable-flow pump, a metering pump, a solenoid valve, and a PLC, where the motor converter is configured to adjust a rotational speed of an agitator; the variable-flow pump and the metering pump are configured to control quantitative feeding in an extraction process; the solenoid valve is configured to control feeding and discharging of a feed solution; and the PLC is configured to obtain a control instruction, and transmit the control instruction to the motor converter, the variable-flow pump, the metering pump, and the solenoid valve.

The process detection module is configured to obtain real-time data.

The process detection module includes a flowmeter, a level gauge, a thermometer, a pH meter, and component content detection devices. The flowmeter is configured to monitor and control a feeding flow rate and a discharging flow rate of the feed solution in an extraction process. The level gauge is configured to monitor and detect levels of liquid in an extraction tank and a storage tank. The thermometer and the pH meter are configured to prepare a scrubbing solution and an extraction solution that meet requirements for temperature and potential of hydrogen. The component content detection devices are disposed for each detection level of a rare earth extraction process.

The virtual workshop M2 for rare earth production is configured to build a virtual rare earth workshop based on a geometric model and a control script of the production site as well as the real-time data, for a user to perform inspection.

The virtual workshop M2 for rare earth production includes: a data exchanging module, a geometric model base, a user interaction module, and a scene changing module.

The data exchanging module is configured to regularly query related data in the twin database according to process-data correspondence.

The geometric model base is configured to build a virtual rare earth workshop, and visualize the virtual rare earth workshop, where the virtual rare earth workshop is built by using modeling software.

The user interaction module is configured to control a scene viewing angle when the user performs inspection, and a demo animation.

The scene changing module is configured to change a virtual rare earth workshop when the user performs inspection.

The digital twin service system M3 is configured to:

obtain historical data of a process production index;

optimize an extraction mechanism model based on the historical data of the process production index according to a parameter optimization algorithm, to obtain a data-driven model, where the extraction mechanism model is a mathematical model representing an extraction mechanism of rare earth;

obtain process information input by the user;

forecast a content of each element in a finished product of a process based on the process information by using the data-driven model; and

determine, based on the content of each element, whether to give a warning.

In some examples, the digital twin service system further includes a process optimization module configured to:

obtain an optimization strategy by using an optimal control algorithm based on an objective set by the user, where the optimal control algorithm is used for optimal control over a flow rate of a reagent in extraction based on static setting and dynamic compensation.

That the control information system M1 for rare earth production is configured to control a production process may be control the production process according to the optimization strategy. The optimization strategy is used as a control instruction that is executed by the basic control module.

The digital twin service system in this example may further include a model updating module configured to:

calculate an error between the forecast content and actual data of a process index, to obtain a content error; and adjust the data-driven model based on the content error according to the parameter optimization algorithm.

In addition, the system for virtual inspection and simulation of a rare earth production process provided in this example further includes: a twin database. The twin database is a bridge between virtual data and real data, and is configured to store various data, including but is not limited to: historical data, real-time data, and warning information.

To exchange date with the twin database, the control information system for rare earth production further includes: a data transmission module.

### EXAMPLE 3

This example provides a digital twin-based system for virtual inspection and simulation of a rare earth production process, and provides a technical solution capable of implementing centralized monitoring and controlling of process indexes and simulating and forecasting a process index to resolve such a problem that device inspection, working condition determining, and process optimization in a rare earth production process rely heavily on manual work.

The digital twin-based system for virtual inspection and simulation of a rare earth production process provided in this example includes a control information system for rare earth production, a twin database, a virtual workshop for rare earth production, and a digital twin service system.

All modules exchange data through the twin database. The control information system for rare earth production stores collected real-time data into the twin database and reads an optimization strategy from the twin database. The virtual workshop for rare earth production reads the data in the twin database in a real-time manner, and sends an actual device status, a process index, and a forecast value of a component content to the virtual workshop. The digital twin service system can not only query the data in the twin database, but also store the forecast value of the component content and the optimization strategy into the twin database. The control information system for rare earth production is mainly configured to deliver a device control instruction and collect on-site detection data to form real-time data of the process index; and store the real-time data into the twin database.

The twin database is configured to store real-time data and process index historical data that are collected at a site, as well as control strategy data and user management information that are optimized. These pieces of data can be queried by a user and used to optimize a forecasting model.

The virtual workshop for rare earth production is mainly configured to implement inspection of a production device and monitoring and controlling of a production index. After being built, the virtual workshop for rare earth production is integrated into the digital twin service system.

The digital twin service system is mainly configured to implement process index forecasting, process optimization, virtual workshop inspection, and query for device data and process data. Finally, the above functions are integrated into a user interface to facilitate user's operation.

According to the digital twin-based system for virtual inspection and simulation of a rare earth production process, the control information system for rare earth production includes a basic control module, a process detection module, a data transmission module, an optimal control module, and a centralized control module. The basic control module and the process detection module are mainly disposed at a production site. The data transmission module, the optimal control module, and the centralized control module are deployed in a central control room. The basic control module is mainly configured to execute a control instruction delivered by the centralized control module. The process detection module is mainly configured to detect a device status and a process index. The data transmission module transmits collected real-time detection data to the centralized control module. The optimal control module and the centralized control module are mounted in a computer in a form of software. The centralized control module may upload real-time data through the data transmission module. The optimal control module reads an optimization strategy from the twin database through the data transmission module, performs certain conversion on the optimization strategy, and transfers a converted optimization strategy to the centralized control module. Then, the centralized control module delivers settings of the optimal control module to the basic control module.

The basic control module is configured to implement loop and quantitative control of a production device, and execution of an optimization strategy, and mainly includes a motor converter, a variable-flow pump, a metering pump, a solenoid valve, and a PLC. The motor converter is configured to adjust a rotational speed of an agitator. The variable-flow pump and the metering pump are configured to implement quantitative feeding in an extraction process. The solenoid valve is configured to control feeding and discharging of a feed solution. The PLC converts a control instruction obtained from the centralized control module into an electrical signal, and transfers the electrical signal to each actuator. The basic control module executes control instructions obtained from the centralized control module and the optimal control module, thereby automatically controlling the production process.

The process detection module is configured to detect a production index, collect data, and send the data to the centralized control module through the data transmission module, thereby implementing collection and management of on-site data. The data collected by the process detection module is the above real-time data. The process detection module mainly includes a flowmeter, a level gauge, a thermometer, a pH meter, and component content detection devices. The flowmeter is configured to monitor and control a feeding flow rate and a discharging flow rate of the feed solution in an extraction process. The level gauge is configured to monitor and detect levels of liquid in an extraction tank and a storage tank, to keep the levels normal. The thermometer and the pH meter are configured to prepare a scrubbing solution and an extraction solution that meet process requirements. The component content detection devices may be x-ray fluorescence analyzers, and are disposed for each detection level of a rare earth extraction process. Each detection device uploads detection data to the centralized control module through a transmitter and the data transmission module. The centralized control module transmits the data from the detection device in a real-time manner to the data transmission module for processing.

The data transmission module is mainly configured to help the process detection module and the centralized control module implement data transmission and data cleaning of on-site detection data, help the twin database and the control information system for rare earth production implement exchange data, and provide various interfaces for on-site detection devices to communicate with a twin database in a server in a TCP/IP manner at the same time.

The optimal control module is mainly configured to convert data of an optimization strategy in the twin database into an XMAL file, and send the XMAL file to the centralized control module for optimal control of an effect. The optimal control module reads the data of the optimization strategy in the twin database. The optimization strategy is generated by the process optimization module of the digital twin service system, and is stored in a twin database. The optimal control module is mainly used as middleware between the centralized control module and the twin database.

The centralized control module is mainly configured to implement centralized control over each device, changing of a specified value of a controller, and centralized control over data collected at a site; may be implemented through configuration software; may communicate with the twin database through the data transmission module; and may communicate with the basic control module and the process detection module that are of a lower layer.

According to the digital twin-based system for virtual inspection and simulation of a rare earth production process, the twin database includes a user management information base, an optimization strategy base, a historical process index database, a real-time database, and a warning database; and may be implemented by a Mysql database of the server.

The user management information base is configured to: store user historical operation data, operator log information, and device maintenance information; and implement recording and traceback of production operations.

The real-time data base is configured to store real-time detection data and control information that are collected by the control information system for rare earth production. Data in the database is a key to implement virtual reality (VR) interaction. The database may refresh data based on a specified detection period, and copy outdated data to the historical process index database,

The process index historical database is mainly configured to store previous real-time data. Such data may be further used to optimize a data-driven model, and may be used by a user for traceback.

The warning information base is configured to store process index data of an abnormal working condition. Warning information includes not only warning for a real-time working condition but also warning for a device status; is generated by the control information system for rare earth production; and is displayed in the digital twin service system for rare earth production and a virtual rare earth workshop.

According to the digital twin-based system for virtual inspection and simulation of a rare earth production process, the virtual workshop for rare earth production includes a data exchanging module, a geometric model base, a user interaction module, and a scene changing module. The virtual workshop for rare earth production may be implemented by using scene building software such as Unity and UE4.

The data exchanging module is configured to implement data exchanging between the virtual workshop and the twin database, and between the virtual workshop and the digital twin service system, thereby facilitating data visualization for the workshop. The data exchanging module is mainly configured to regularly query related data in the twin database according to a one-to-one correspondence between process and data.

The geometric model base mainly stores a three-dimensional model of an actual workshop; is configured to build the virtual workshop for rare earth production, thereby visualizing the process; and may be built based on an actual production device by using modeling software such as 3DMAX and MAYA.

The user interaction module is configured to enable a user to perform free inspection in a virtual scene and to view process data and device information; and may implement a user inspection panel, a process demo animation, and control of a scene viewing angle by using a script writing program.

The scene changing module is configured to enable the user to move in different virtual production workshops, such that the user can inspect all workshops fast. The scene changing module performs scene changing and initializes data in each scene by using a script program.

According to the digital twin-based system for virtual inspection and simulation of a rare earth production process, the digital twin service system mainly includes a process index forecasting module, a virtual workshop inspection module, a device information query module, a forecasting algorithm, a process optimization algorithm, a process optimization module, a model updating module, and a user interface.

The process index forecasting module includes a process mechanism model, a data-driven model, and a real-time forecasting model. The process mechanism model is a mathematical model built according to a process principle, and includes models of separation coefficients between extraction stages (for example, formulas (1)-(3)), specific models for conservation of materials and the like (for example, formula (4)-(7)), and models of parameters for compensating a separation coefficient (for example, formula (8)-(9)). The data-driven model is to adjust a separation coefficient of a model according to historical process data and the model updating module to further improve precision of the model. A principle of the real-time forecasting model is similar to that of the data-driven model. A difference between the two principles lies in that the real-time forecasting model performs forecasting after a model is adjusted based on recent real-time data. The user inputs basic process information of a site, and forecasts a process index of each production link in a next inspection cycle with reference to the real-time data in the twin database by using the real-time forecasting model. The process index forecasting module sends a forecast result to the twin database and the process optimization module. Separation coefficient models are shown below. X denotes a content of an aqueous component. Y denotes a content of an organic component. Formula (1) denotes a separation coefficient for two adjacent rare earth elements. Formula (2) denotes a separation coefficient of an extraction element relative to a first element. Formula (3) denotes a separation coefficient of the extraction element relative to a last element. Conservation of materials between extraction stages is used to describe conservation of contents of extraction elements in two adjacent extraction stages. f_{F} in formula (4) of a conservation of materials model denotes a content of the feed solution. f′_{B} denotes an output mole fraction of a hardly extractable component. f̅′_{A} denotes an output mole fraction of an easily extractable component. An organic composition of output component A and an aqueous composition of output component B are obtained by dividing f̅′_{A} and f̅′_{B} into a content of output component A and a content of output component B, respectively. S̅ denotes an extraction capacity. W denotes a scrubbing capacity. i denotes the number of an extracted element. i ranges from 1 to N. N denotes the number of an element that is extracted last. Yi denotes a content of an organic component of the i^{th} element. Xi denotes a content of an aqueous component of the i^{th} element. β_{(1+i)/i} denotes a separation coefficient of an element relative to a next element. β_{1/i} denotes a separation coefficient of the i^{th} element relative to the first element. β_{i/N⋅} denotes a separation coefficient of the last element relative to the i^{th} element. X_{[k+1,i]} denotes a content of an aqueous component that is of a (k+1)^{th} level and of the i^{th} element. Y_{[k,i]} denotes a content of an aqueous component that is of a k^{th} level and of the i^{th} element. A conservation of materials relationship of the hardly extractable component may be established among a product of the output mole fraction of the hardly extractable component f′_{B} and a content of an aqueous component, contents of aqueous and organic components of a feeding level, the extraction capacity, and the scrubbing capacity. Similarly, a conservation of materials relationship of the easily extractable component may be established among a product of the output mole fraction of the easily extractable component f̅′_{A} and a content of an organic component, a content of aqueous and organic components of a feeding level, the extraction capacity, and the scrubbing capacity. Contents of components of all levels can be deduced by level according to these conservation of materials relationships. A case in which on-site extraction is insufficient can be simulated by adding a compensation parameter K to a separation coefficient. Then, a relationship between an aqueous component and an organic component can be obtained by performing deformation. A content of one of the two components can be obtained based on a content of the other component.

Formula (1) denotes a relationship between an organic component content and a separation coefficient for two adjacent elements. Formula (2) denotes a separation coefficient of an i^{th} element relative to a first element. Formula (3) denotes a separation coefficient of the i^{th} element relative to a last element. Formulas (4 and 5) denote relationships between aqueous components of two adjacent levels in an extraction stage. Formulas (6 and 7) denote relationships between organic components of two adjacent levels in a scrubbing stage. Formulas (8 and 9) denote relationships between an aqueous component and an organic component that are of a same level and have been compensated based on a separation coefficient.

$${β}_{( i + 1 ) / i} = \frac{{Y}_{i + 1} \times {X}_{i}}{{Y}_{i} \times {X}_{i + 1}} \qquad ( 1 )$$

$${β}_{1 / i} = \frac{{Y}_{i} \times {X}_{1}}{{Y}_{1} \times {X}_{i}} \qquad ( 2 )$$

$${β}_{i / N} = \frac{{Y}_{N} \times {X}_{i}}{{Y}_{i} \times {X}_{N}} \qquad \text{(3)}$$

$${f}_{B}^{′} \times {X}_{[ 1 , i ]} = W \times {X}_{[ k + 1 , i ]} - \overline{S} \times {Y}_{[ k , i ]} , i = 1 , 2 , … , N ; k = 1 , 2 , … n \qquad ( 4 )$$

$${X}_{[ k + 1 , i ]} = ( \overline{S} \times {Y}_{[ k , i ]} + {f}_{B}^{′} \times {X}_{[ 1 , i ]} ) / W \qquad ( 5 )$$

$${\overline{f}}_{A}^{′} \times {Y}_{[ n + m , i ]} = ( \overline{S} + 1 ) \times {Y}_{[ k + 1 , i ]} - W \times {X}_{[ k , i ]} , \qquad ( 6 )$$

$$i = 1 , 2 , … , N ; k = 1 , 2 , … , m$$

$${Y}_{[ k + 1 , i ]} = ( W \times {X}_{[ k , i ]} + {\overline{f}}_{A}^{′} \times {Y}_{[ n + m , i ]} ) / ( \overline{S} + 1 ) \qquad ( 7 )$$

$${Y}_{i} = ( {X}_{i} \times {\prod }_{i = 1}^{N} K . * {β}_{1 / i} ) / {\sum }_{i = 1}^{N} ( {X}_{i} \times {\prod }_{i = 1}^{N} K . * {β}_{1 / i} ) \qquad \text{(8)}$$

$${X}_{i} = ( {Y}_{i} \times {\prod }_{i = 1}^{N} K . * {β}_{i / N} ) / {\sum }_{i = 1}^{N} ( {Y}_{i} \times {\prod }_{i = 1}^{N} K . * {β}_{i / N} ) \qquad ( 9 )$$

The virtual workshop inspection module integrates the virtual rare earth workshop into a sub-interface of the user interface, such that the user can view the virtual workshop conveniently. The virtual workshop inspection module is implemented based on the virtual rare earth workshop; and needs to receive an operation of the user in the virtual workshop and implement connection with other modules.

The device information query module is mainly configured to read the real-time process index, the device status, and the warning information in the twin database, such that the user can query information fast as required. The device information query module may use an SQL statement to implement a query function for the twin database. A fuzzy query mode may be designed to help the user query information. An input of the device information query module is a process link, a device number, a process index, or a time stamp that is specified by the user. Output information of the device information query module is a process index, a device status, or a process index forecast result.

The forecasting algorithm is mainly used to build the mathematical model based on an extraction mechanism offline; then, obtain the data-driven model according to the process index historical data and the parameter optimization algorithm; and finally forecast contents of three elements Ce, Pr, and Nd based on the process information input by the user, and provide over-limit warning information. The data-driven model is configured to optimize a separation coefficient of the mechanism model according to the parameter optimization algorithm; and calculate an optimal compensation parameter for the separation coefficient based on historical data of contents of components of all levels. The data-driven model is mainly configured to read historical data, optimize a compensation parameter, and output a forecast result. The data-driven model calculates by level a content of a component of each level according to specified feeding and discharging manners, a content of an initial component of the feeding level, and an optimized mechanism model. The parameter optimization algorithm is specifically used to optimize the separation coefficient in the mechanism model according to the mechanism model and component content data. An optimization manner is optimizing the compensation parameter K of the separation coefficient according to an improved particle swarm optimization algorithm, to obtain an optimal compensation parameter.

The process optimization module is configured to obtain a best optimization strategy according to the optimization algorithm based on an optimization objective specified by the user. The optimization algorithm mainly refers to an optimal control algorithm. Herein, the optimization algorithm refers to a method for optimal control over a flow rate of a reagent in extraction based on static setting and dynamic compensation. The method mainly includes the following steps: first, setting a feeding flow rate of the reagent according to a process; then, adjusting the feeding flow rate of the reagent according to a detected component content, to generate an optimized feeding flow rate; setting and forming a control strategy; and sending the control strategy to the twin database.

The model updating module is configured to adjust a compensation parameter of a model according to the model optimization algorithm and an actual process index, to make the model closer to an actual process condition. A main implementation principle is to use an error between a model forecast result and data of the actual process index as an input, and adjust the compensation parameter of the model according to the optimization algorithm, thereby improving forecasting precision of the model.

The user interface integrates other modules of the digital twin service system, to help the user perform an operation. The user interface is mainly configured to implement communication between each module and the twin database and between each module and the virtual rare earth workshop, and building of the user interface.

Compared with the prior art, the present disclosure has the following positive effects.

According to the present disclosure, based on a real-time process index forecasting model built in a digital twin system, real-time and precise forecasting of a process index can be implemented by using a real-time process index forecasting module. In addition, a response is given fast to a change of a production condition, and warning and optimizing data is provided for the system, such that a burden of an operator is reduced, control over a production process is optimized, production and management efficiency is improved, and process data of the site is utilized effectively.

According to the present disclosure, visualized demonstration of data in each production process is realized by building a virtual rare earth workshop and establishing a data connection between an actual workshop and the virtual rare earth workshop. In addition, fast inspection of a production device is realized.

A control strategy is optimized based on an optimization objective specified by the user and actual process index data by using a process optimization module in a digital twin service system, thereby optimizing the production process.

### EXAMPLE 4

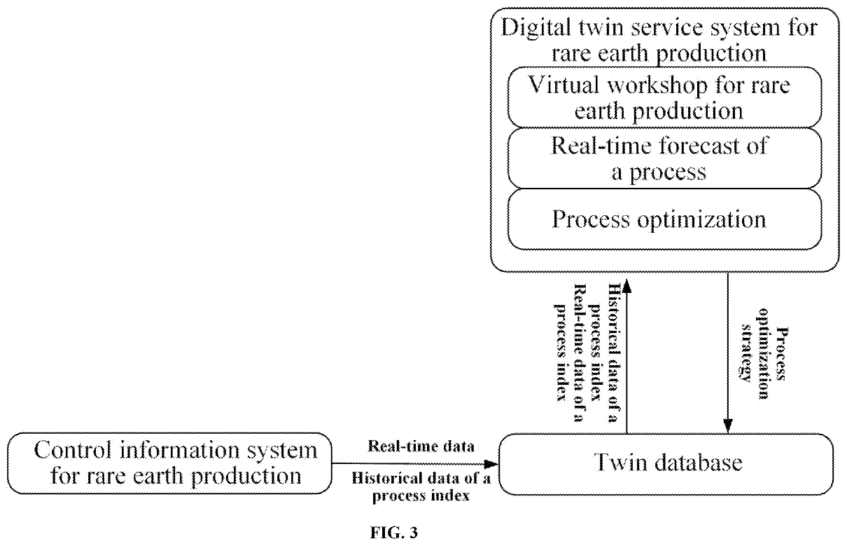

This example provides a digital twin-based system for virtual inspection and simulation of a rare earth production process. As shown in FIG. 3, The system includes a control information system for rare earth production, a twin database, and a rare earth digital twin service system. The control information system for rare earth production is configured to: detect and control a process at a site; generate real-time data of a process index; and execute an optimal control strategy. The twin database is configured to store real-time process index data of the control information system for rare earth production, and an optimization strategy and process index forecasting data of the rare earth digital twin service system. The data is stored in a classified manner according to service requirements of all modules. The rare earth digital twin service system may use actual process data and historical process data for optimization of a real-time process index forecasting model, inspection for a virtual workshop, fast query of production device data, and generation of an optimization strategy.

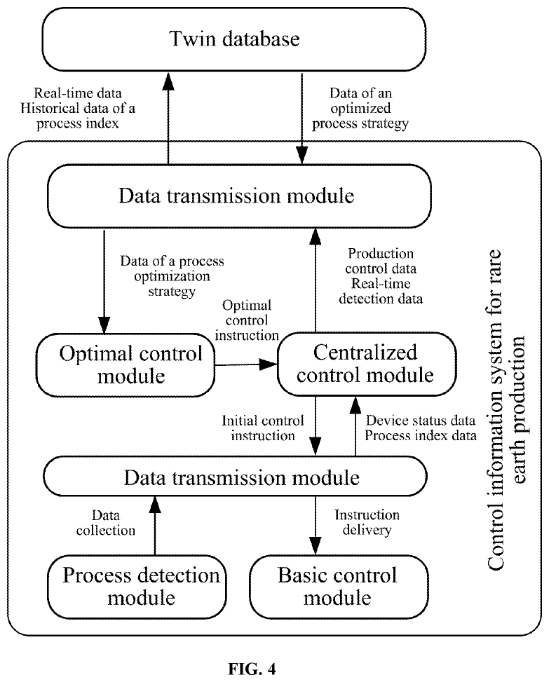

As shown in FIG. 4, the control information system for rare earth production includes a basic control module, a process detection module, a data transmission module, an optimal control module, and a centralized control module.

The basic control module is configured to implement a basic control function of a process, and execute an initial control instruction and the optimization strategy; mainly includes a controller and a corresponding actuator; and specifically includes a PLC, a metering pump, a variable-flow pump, a motor converter, and the like.

The process detection module collects real-time data of a process index, and stores the real-time data into the twin database through the data transmission module. Specifically, there are the following detection devices: a flow meter, a pH meter, a thermometer, a level gauge, and component content detection devices. The data transmission module is configured to implement connection among the detection devices, a control device, and the centralized control module, connection among the centralized control module, the optimal control module, and the twin database. Specifically, the connection may be implemented by using industrial Ethernet or profibus. The data transmission module transmits data to the twin database according to the OPCUA or TCP/IP protocol. The optimal control module is configured to transmit optimization strategy data into a control instruction. The centralized control module is mainly configured to specify a site control parameter and monitor and control detection information, and may be implemented through configuration software.

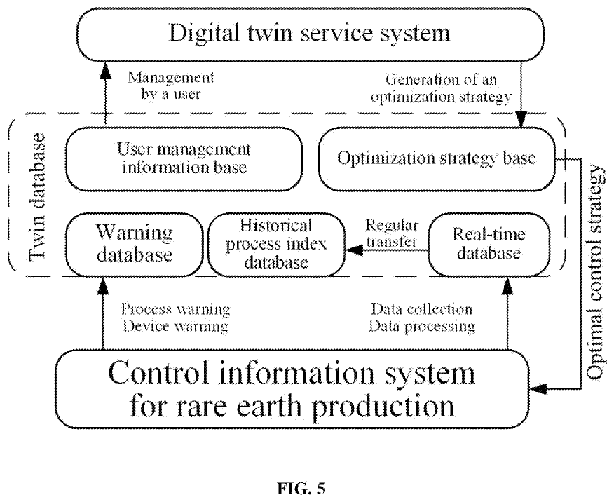

As shown in FIG. 5, the twin database includes a user management information base, an optimization strategy base, a real-time process index database, a historical process index database, and a warning database.

The user management information is mainly configured to record a user operation and user login information. The optimization strategy base is configured to store an optimization strategy generated by the twin database. The real-time data base is configured to store the real-time process index data collected by the control information system for rare earth production. The process index historical database stores real-time data transferred regularly. The warning database stores overrun warning data sent by the control information system for rare earth production. The twin database is implemented by using a Mysql database.

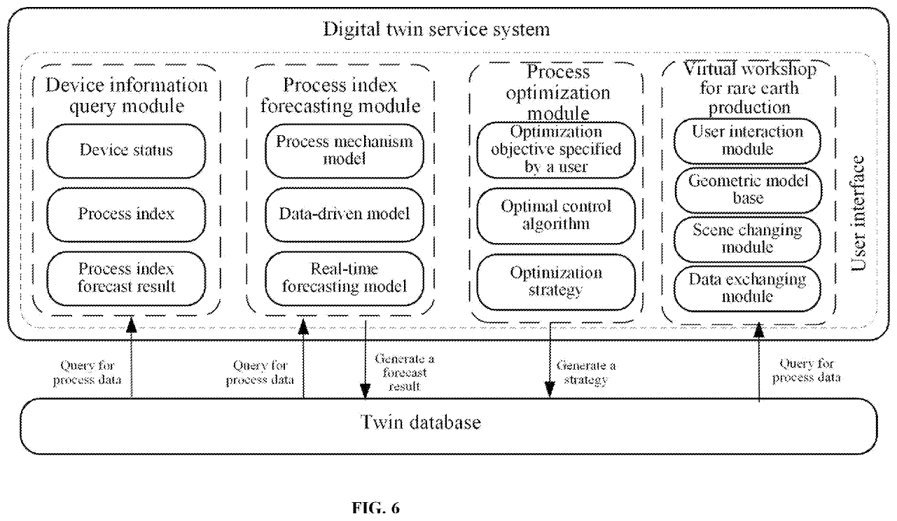

As shown in FIG. 6, the rare earth digital twin service system includes a device information query module, a process index forecasting module, a process optimization module, a virtual workshop for rare earth production, and a user interface.

The device information query module is mainly configured to implement fast query and retrieval function for data in the twin database; and mainly queries process data by using an SQL statement. The process optimization module is configured to optimize a control strategy based on an optimization algorithm, a specified optimization objective, and actual process data. The process index forecasting module builds a real-time forecasting model, and then forecasts a process index data based on real-time data. By building a virtual workshop scene, the virtual workshop for rare earth production implements visualization of a production process, and virtual inspection for a workshop device. The virtual workshop for rare earth production may be implemented by using scene building software Unity and modeling software 3Dmaxs. Data consistency of the virtual workshop is guaranteed by searching the twin database for process data. The user interface integrates all the modules.

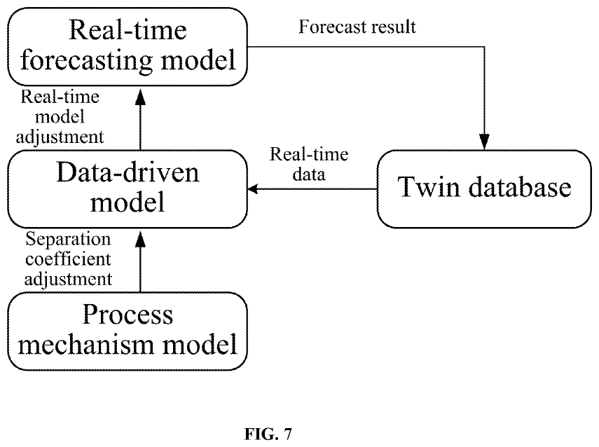

As shown in FIG. 7, the real-time process index forecasting module includes a mechanism model, a data-driven model, and a real-time forecasting model. First, the mechanism model was built by using a separatory funnel method for rare earth extraction; then, an error between process index historical data and a forecast value was read from the twin database; a best separation coefficient was obtained by correcting a separation coefficient according to a differential evolution algorithm; and finally, a supplementary data-driven model was built for a rare earth extraction process. To further guarantee precision of a model, real-time data in the twin database was compared with a forecast value of the data-driven model; and then, the differential evolution algorithm was used again to further adjust the separation coefficient, thereby obtaining a more precise forecasting model.

According to the digital twin-based system for virtual inspection and simulation of a rare earth production process, a building process of the process index forecasting module includes the following steps:

(1) Build a process mechanism model by using the separatory funnel method for rare earth extraction.

(2) The data-driven model is obtained by adjusting a separation coefficient of the process mechanism model based on historical process index data and a model optimization algorithm, to make the model more precise.

(3) The real-time forecasting model is obtained by dynamically adjusting an offline model such as the process mechanism model and the data-driven model based on recent real-time data, to decrease an error of the model as far as possible.

(4) Finally, real-time forecasting models are packaged, and then integrated into the user interface.

According to the digital twin-based system for virtual inspection and simulation of a rare earth production process, a building process of the virtual rare earth workshop includes the following steps:

(1) Build a three-dimensional model of each device according to a production process, store the three-dimensional model into a geometric model base.

(2) Visualize the production process by using the scene building software.

(3) Write scripts to implement user's viewing angle control, data communication, and display of a human-computer interaction interface.

(4) Finally, virtual scenes are packaged and then integrated into the user interface.

The process index data includes a content of a detection-level component, a speed of a stirring motor, a temperature in a stirring tank, a liquid level, a pH value, a flow rate of a detergent, a flow rate of an extraction agent, and a feeding flow rate of a raw material.

A running method for the digital twin-based system for virtual inspection and simulation of a rare earth production process provided in the present disclosure includes the following steps:

(1) Data collection and device control: When a production process is running normally, a device is automatically controlled according to initial control settings, and the control information system for rare earth production at a production site collects process production index data of the production site, and sends the data to the twin database.

(2) Data storage and classification: The twin database is implemented by using a database file in a server, and is mainly configured to store real-time process index data and process warning data that are connected at the site. A twin service system for rare earth optimizes strategy data, and classifies data according to requirements of different modules and process types, to obtain a data table that is more convenient to search.

(3) Virtual scene inspection: The virtual rare earth workshop is built by using the virtual scene building software based on a device geometric model and a control script. A one-to-one correspondence between virtual scenes and real scenes is established by reading data in the twin database, such that a user can freely view a production process status by performing operation.

(4) Simulation and process optimization for rare earth production: A rare earth digital twin service platform reads real-time process index data in the twin database and uses the data for real-time process index forecasting, to simulate a rare earth production process; and then stores an obtained process index forecast result in the twin database for other modules to use. Process optimization is to use historical process data, a process optimization algorithm, and an optimization objective set by the user to optimize settings of a controller; and then, store an optimized control strategy into the twin database, to facilitate execution of the control information system for rare earth production.

Each example of this specification is described in a progressive manner, each example focuses on a difference from other examples, and the same and similar parts between the examples may refer to each other.

In this specification, some specific examples are used for illustration of the principles and implementations of the present disclosure. The description of the foregoing examples is used to help illustrate the method of the present disclosure and the core ideas thereof. In addition, persons of ordinary skill in the art can make various modifications in terms of specific implementations and the scope of application according to the ideas of the present disclosure. In conclusion, the content of the present description shall not be construed as limitations to the present disclosure.

What is claimed is:

1. A method for virtual inspection and simulation of a rare earth production process, comprising:
obtaining real-time data of a production site, wherein the real-time data comprises data of a process production index;
building a virtual rare earth workshop based on a geometric model and a control script of the production site as well as the real-time data, for a user to perform inspection;
obtaining historical data of the process production index;
optimizing an extraction mechanism model based on the historical data of the process production index according to a parameter optimization algorithm to obtain a data-driven model, wherein the extraction mechanism model is a mathematical model representing an extraction mechanism of rare earth;
obtaining process information input by the user;
forecasting a content of each element in a finished product of a process based on the process information by using the data-driven model; and
determining, based on the content of each element, whether to give a warning.

2. The method for virtual inspection and simulation of a rare earth production process according to claim 1, wherein the optimizing an extraction mechanism model based on the historical data of the process production index according to a parameter optimization algorithm to obtain a data-driven model specifically comprises:
optimizing a separation coefficient in the extraction mechanism model based on the historical data of the process production index according to a particle swarm optimization algorithm, to obtain the data-driven model.

3. A system for virtual inspection and simulation of a rare earth production process, comprising: a control information system for rare earth production, a virtual workshop for rare earth production, and a digital twin service system, wherein
the control information system for rare earth production is configured to obtain real-time data of a production site, and control a production process;
the virtual workshop for rare earth production is configured to build a virtual rare earth workshop based on a geometric model and a control script of the production site as well as the real-time data, for a user to perform inspection; and
the digital twin service system is configured to:
obtain historical data of a process production index;
optimize an extraction mechanism model based on the historical data of the process production index according to a parameter optimization algorithm to obtain a data-driven model, wherein the extraction mechanism model is a mathematical model representing an extraction mechanism of rare earth;
obtain process information input by the user;
forecast a content of each element in a finished product of a process based on the process information by using the data-driven model; and
determine, based on the content of each element, whether to give a warning.

4. The system for virtual inspection and simulation of a rare earth production process according to claim 3, wherein the control information system for rare earth production comprises a basic control module and process detection modules, wherein
the basic control module is configured to execute a control instruction;
the basic control module comprises a motor converter, a variable-flow pump, a metering pump, a solenoid valve, and a PLC, wherein the motor converter is configured to adjust a rotational speed of an agitator; the variable-flow pump and the metering pump are configured to control quantitative feeding in an extraction process; the solenoid valve is configured to control feeding and discharging of a feed solution; and the PLC is configured to obtain a control instruction, and transmit the control instruction to the motor converter, the variable-flow pump, the metering pump, and the solenoid valve;
the process detection module is configured to obtain real-time data; and
the process detection module comprises a flowmeter, a level gauge, a thermometer, a pH meter, and component content detection devices, wherein the flowmeter is configured to monitor and control a feeding flow rate and a discharging flow rate of the feed solution in an extraction process; the level gauge is configured to monitor and detect levels of liquid in an extraction tank and a storage tank; the thermometer and the pH meter are configured to prepare a scrubbing solution and an extraction solution that meet requirements for temperature and potential of hydrogen; and the component content detection devices are disposed for each detection level of a rare earth extraction process.

5. The system for virtual inspection and simulation of a rare earth production process according to claim 3, further comprising:
a twin database configured to store historical data, real-time data, and warning information.

6. The system for virtual inspection and simulation of a rare earth production process according to claim 5, wherein the control information system for rare earth production further comprises a data transmission module configured to exchange data between the twin database and the control information system for rare earth production.

7. The system for virtual inspection and simulation of a rare earth production process according to claim 5, wherein the virtual workshop for rare earth production comprises: a data exchanging module, a geometric model base, a user interaction module, and a scene changing module, wherein
the data exchanging module is configured to regularly query related data in the twin database according to process-data correspondence;
the geometric model base is configured to build a virtual rare earth workshop, and visualize the virtual rare earth workshop, wherein the virtual rare earth workshop is built by using modeling software;
the user interaction module is configured to control a scene viewing angle when the user performs inspection, and a demo animation; and
the scene changing module is configured to change a virtual rare earth workshop when the user performs inspection.

8. The system for virtual inspection and simulation of a rare earth production process according to claim 3, wherein the digital twin service system further comprises: a process optimization module configured to:
obtain an optimization strategy by using an optimal control algorithm based on an objective set by the user, wherein the optimal control algorithm is used for optimal control over a flow rate of a reagent in extraction based on static setting and dynamic compensation.

9. The system for virtual inspection and simulation of a rare earth production process according to claim 8, wherein the controlling a production process specifically comprises:
controlling the production process according to the optimization strategy.

10. The system for virtual inspection and simulation of a rare earth production process according to claim 3, wherein the digital twin service system further comprises a model updating module configured to:
calculate an error between the forecast content and actual data of a process index, to obtain a content error; and
adjust the data-driven model based on the content error according to the parameter optimization algorithm.
